(19) United States
(12) Patent Application Publication
(10) Pub. No.: US 2025/0177605 A1
(43) Pub. Date: Jun. 5, 2025

(54) BONE FILLING MATERIAL WITH TRACE ELEMENTS

(71) Applicant: NATIONAL UNIVERSITY OF KAOHSIUNG, Kaohsiung (TW)

(72) Inventors: Wen-Fu Ho, Kaohsiung (TW); Peng-Hsiang Wang, Kaohsiung (TW)

(73) Assignee: NATIONAL UNIVERSITY OF KAOHSIUNG, Kaohsiung (TW)

(21) Appl. No.: 18/402,309

(22) Filed: Jan. 2, 2024

(30) Foreign Application Priority Data
Nov. 30, 2023 (TW) 112146525

(51) Int. Cl. A61L 27/10; A61L 27/04; A61L 27/56

(52) U.S. Cl. CPC A61L 27/10; A61L 27/047; A61L 27/56; A61L 2300/102; A61L 2300/112; A61L 2430/02

(57) ABSTRACT

The present invention provides a calcium phosphate ceramic with trace elements, wherein a main component of the calcium phosphate ceramic is selected from a group consisting of hydroxyapatite (HA), α-tricalcium phosphate (α-TCP), β-tricalcium phosphate (β-TCP), tetracalcium phosphate (TTCP) and any combination thereof; the calcium phosphate ceramic comprises various trace elements, at least including Mg, Sr, Na and Fe. The calcium phosphate ceramic is high biocompatible and non-cytotoxic, meanwhile demonstrates extraordinary features of osteoinductivity, osteoconductivity, osteogenesis activity and achieves the purpose of bone repair.

### CROSS-REFERENCE TO RELATED APPLICATIONS

This application claims the benefit of priority to Taiwan Patent Application No. 112146525, filed on Nov. 30, 2023, which is incorporated by reference herein in its entirety.

### BACKGROUND

The present invention provides a bone filling material with various trace elements and its application. More specifically, the bone filling material has at least Mg, Sr, Na and Fe, ideal biological characteristics such as biological activity, biocompatibility, osteoinductivity, osteoconductivity, osteogenesis.

Calcium phosphate ceramic provides excellent biological activity, biocompatibility and biological absorbability. Therefore, it is commonly used as biomedical material, such as in orthopedics and tooth filling, drug carrier and scaffold for tissue engineering. Commonly seen calcium phosphate ceramic includes hydroxyapatite (HA), β-tricalcium phosphate (β-TCP), α-tricalcium phosphate (α-TCP), etc. The most representative calcium phosphate ceramic is HA [Ca₁₀(PO₄)₆(OH)₂]. HA has a calcium to phosphorus stoichiometric ratio of 1.67, is the main mineral component of human bone, and has favorable bioactivity, biocompatibility, and osteoconductivity.

The bone filling materials currently used clinically are osteoconductive but not osteoinductive. Therefore, their low success rate is their biggest disadvantage. Osteoconductivity of the bone filling material accounts for cell migration and adhesion. In addition, bone growth factors are also crucial to bone damage areas during bone healing process. Clinically the addition of bone growth factors, such as bone morphogenetic protein (BMP), can make bone filling materials osteoinductive. However, growth factors are expensive. In addition, off label usage and adverse events such as postoperative complications have become concerns. For example, severe side effects of BMP-2 include edema, erythema, and ectopic bone formation, etc, which causes clinical application to be limited.

Comparing to adding growth factors to bone filling materials, adding trace elements in bone filling materials is considered a more natural and safer method. It can lower costs, extend shelf life and lower risks. However, there still exists two problems when incorporating trace elements artificially. Firstly, the process of incorporating multiple types of trace elements is complicated. It is difficult to avoid other impurity ions being incorporated that will alter the original composition and structure of the bone filling material. Secondly, ion concentration is hard to be determined and thus biological safety cannot be guaranteed. Moreover, when adding a single ion, a high concentration is typically required to trigger osteoinduction of bone filling material. However, concerns exist about the biological toxicity at high concentrations, which refrains the bone filling material in commercial use.

### SUMMARY

Due to the aforementioned problems, the present invention aims to provide an ideal bone filling material, including various trace elements. The bone filling material provides good physical and chemical properties for migration and proliferation of bone tissue, including appropriate pore size, porosity and biodegradability, etc. Excellent biological characteristics such as biological activity, biocompatibility, osteoinductivity, osteoconductivity, osteogenesis, osteogenesis are also required in order to promote new bone growth and achieve the results of bone repair.

In order to accomplish the purpose above, the present invention provides the following technical means.

In an aspect, the present invention provides a calcium phosphate ceramic with trace elements, wherein a main component of the calcium phosphate ceramic is selected from a group consisting of hydroxyapatite (HA), α-tricalcium phosphate (α-TCP), β-tricalcium phosphate (β-TCP), tetracalcium phosphate (TTCP) and any combination thereof; the calcium phosphate ceramic comprises various trace elements, at least including Mg and Sr.

In some embodiments, the calcium phosphate ceramic of the present invention comprises at least Mg, Sr, Na and Fe.

The calcium phosphate ceramic of the present invention does not require incorporation of artificial element ions.

In some embodiments, source of the calcium phosphate ceramic of the present invention are from biological waste containing calcium. The biological waste containing calcium includes but not limited to egg shells, the shells of Crustacea animals such as crabs, shrimps, lobsters, crayfishes and krills, the shells of Bivalia animals, such as oysters, mussels, clams, the shells of Gastropod animals, such as abalones.

In some embodiments, the main component of the calcium phosphate ceramic of the present invention is biphasic calcium phosphate or triphasic calcium phosphate. In some embodiments, the main component of the calcium phosphate ceramic of the present invention is biphasic calcium phosphate of hydroxyapatite and β-tricalcium phosphate. In some embodiments, the main component of the calcium phosphate ceramic of the present invention is triphasic calcium phosphate of hydroxyapatite, β-tricalcium phosphate, and α-tricalcium phosphate. When bone grafting material contains β-tricalcium phosphate, its excellent biodegradability can create an oversaturation state of Ca/P on the surface and 3D porous structure of the material. This Ca/P enriched state promotes the deposition of hydroxyapatite, thereby inducing migration of mesenchymal stem cells into the material and osteogenic differentiation and promoting new bone growth.

For example, in bone scaffold material containing HA/β-TCP biphasic calcium phosphate, good biocompatibility and non-degradable properties of hydroxyapatite can provide osteogenesis-related cells with good support, allowing the cells to continuously proliferate and secrete extracellular matrix (i.e. type I collagen) within the pores of the scaffold, and the mineralization of the extracellular matrix occurs in a Ca/P saturated environment, which is formed after the degradation of β-tricalcium phosphate, and ultimately generate new bone tissue. The degraded β-tricalcium phosphate also provides space for the formation of new bone, synchronizing the degradation of the material with the regeneration of bone tissue.

In some embodiments, the calcium phosphate ceramic of the present invention is a biphasic calcium phosphate of hydroxyapatite and β-tricalcium phosphate, wherein the volume ratio of hydroxyapatite to β-tricalcium phosphate can be 90:10 to 50:50, preferably 80:20 to 50:50, more preferably 70:30 to 50:50. In some embodiments, the volume ratio of hydroxyapatite to β-tricalcium phosphate in the calcium phosphate ceramic of the present invention is approximately 65:35, 60:40 or 50:50.

The calcium phosphate ceramic of the present invention has ideal osteoinductivity and osteogenesis activities, and can induce the differentiation of mesenchymal stem cells into osteoblasts to promote osteogenesis.

The calcium phosphate ceramic of the present invention can be prepared using the method disclosed in Taiwan Patent No. 1769746. In some embodiments, egg shells are used as raw material in the present invention. Hydroxyapatite powder is prepared through liquid phase synthesis methods, such as sol-gel method, micro emulsion method, chemical precipitation method, hydrothermal method, microwave radiation method, etc. Next, use pore-forming agent and subsequent sintering process to obtain the biphasic or triphasic calcium phosphate ceramic of the present invention. In some embodiments, pore-forming agent can be selected from a group consisting of polyvinylpyrrolidone (PVP), polylactic co-glycolic acid (PLGA), stearic acid, sucrose and graphite. In some embodiments, for example, the sintering process may include the steps of raising the temperature to 200° C. to 500° C. at a heating rate of 1 to 5 degrees per minute, temperature being maintained for 2 to 5 hours, then the temperature being raised to 1,000° C. to 1,300° C. at a heating rate of 1 to 5 degrees per minute and the temperature being maintained for 5 to 30 hours to obtain the biphasic calcium phosphate material. In some embodiments, for example, the sintering process may include the steps of raising the temperature to 200° C. to 500° C. at a heating rate of 1 to 5 degrees per minute, the temperature being maintained for 2 to 5 hours, then the temperature being raised to 1,000° C. to 1,500° C. at a heating rate of 1 to 5 degrees per minute, the temperature being maintained for 1˜10 hours to obtain the triphasic calcium phosphate material.

In some embodiments, the calcium phosphate ceramic of the present invention contains Mg ions in a content range of 1,000 to 10,000 ppm, preferably 2,500 to 8,000 ppm, further preferably 3,000 to 7,200 ppm, more preferably 3,200 to 5,600 ppm, further more preferably 3,600 to 4,500 ppm. For example, in some embodiments, the Mg ion content of the calcium phosphate ceramic of the present invention is approximately 4,000 ppm.

In some embodiments, the calcium phosphate ceramic of the present invention contains Sr ions in a content range of 100 to 1,000 ppm, preferably 200 to 800 ppm, further preferably 300 to 720 ppm, more preferably 350 to 600 ppm, further more preferably 350 to 500 ppm. For example, in some embodiments, the Sr content of the calcium phosphate ceramic of the present invention is approximately 400 ppm.

In some embodiments, the calcium phosphate ceramic of the present invention contains Na ions in a content range of 300 to 3,000 ppm, preferably 500 to 2,800 ppm, further preferably 700 to 2,000 ppm, more preferably 800 to 1,500 ppm, further more preferably 900 to 1,200 ppm. For example, in some embodiments, the Na content of the calcium phosphate ceramic of the present invention is approximately 1,000 ppm.

In some embodiments, the calcium phosphate ceramic of the present invention contains Fe ions in a content range of 10 to 700 ppm, preferably 10 to 500 ppm, further preferably 10 to 300 ppm, more preferably 10 to 200 ppm, further more preferably 10 to 100 ppm. For example, in some embodiments, the Fe content of the calcium phosphate ceramic of the present invention is 30 to 40 ppm.

The calcium phosphate ceramic of the present invention has high porosity and porous structure. The calcium phosphate ceramic of the present invention has evenly distributed pores, including open pores and closed pores. In some embodiments, the porosity of the calcium phosphate ceramic of the present invention is at least 50%, preferably 50 to 90%, further preferably 60 to 90%, more preferably 70 to 90%. In some embodiments, the porosity of the open pores of the calcium phosphate ceramic of the present invention is at least 30%, preferably at least 40%, and further preferably at least 60%. For example, in some embodiments, the porosity of the calcium phosphate ceramic of the present invention is greater than 80%, and the porosity of the open pores is greater than 70%.

The pores of the porous structure of the bone filling material must be interconnected, and their size should be sufficiently large to accommodate bone cells in order for the bone cells to migrate easily through the scaffold. Porous structure has a larger surface area, which results in the adhesion of more proteins and cells to promote the formation of new bone tissue. In some embodiments, the calcium phosphate ceramic of the present invention has macropores with a pore size greater than 100 μm and micropores with a pore size less than 100 μm. The macropore structure provides space for cell growth, and the micropore structure helps the circulation of body fluids. The pore size must be at least greater than 100 μm to allow cell migration. Pore size greater than 300 μm can promote angiogenesis and the formation of new bone tissue.

Moreover, the calcium phosphate ceramic of the present invention also contains many pores with a pore size of less than 10 μm, which gives the calcium phosphate ceramic of the present invention a larger surface area, increases protein adsorption, ion exchange and the formation of mineralized tissue, and provide nutrition and osteogenesis-related signaling molecules to facilitate interaction between cells and the bone filling material, and promote cell proliferation and accelerate bone growth.

In some embodiments, the pore size of the calcium phosphate ceramic of the present invention ranges from 0 to 1,000 μm, preferably from 0 to 900 μm. Illustratively, the pore size of the calcium phosphate ceramic of the present invention may range from 0 to 850 μm. Illustratively, the pore size of the calcium phosphate ceramic of the present invention may range from 0 to 800 μm. In some embodiments, at least 70% of the pores in the calcium phosphate ceramic of the present invention have a pore size greater than 100 μm, preferably at least 75% of the pores have a pore size greater than 100 μm. For example, approximately 80% of the pores have a pore size greater than 100 μm. In some embodiments, at least 60% of the pores of the calcium phosphate ceramic of the present invention have a pore size greater than 300 μm. For example, approximately 65% of the pores have a pore size greater than 300 μm.

In another aspect, the present invention provides a bone filling material, comprising the aforementioned calcium phosphate ceramic.

In another aspect, the present invention provides a use of the aforementioned calcium phosphate ceramic, which is characterized in that the aforementioned calcium phosphate ceramic is used to prepare dental repair materials, orthopedic repair materials, drug carrier materials or tissue engineering scaffolds.

The calcium phosphate ceramic with trace elements of the present invention has high biocompatibility and no cytotoxicity. It also exhibits excellent properties such as osteoconductivity, osteoinductivity and promotion of osteogenesis, thereby achieving the purpose of repairing bone defects. Moreover, the calcium phosphate ceramic of the present invention does not contain heavy metal ions. It complies with the requirement of ASTM F1581-08 of the American Society for Testing and Materials.

The calcium phosphate ceramic with trace elements of the present invention has ideal osteoinductivity and osteogenesis activities, it can induce mesenchymal stem cells to differentiate into osteoblasts, and achieve the effect of promoting osteogenesis. Those with trace elements comparing with bone transplant materials that do not contain trace elements, the bone growth rate of calcium phosphate ceramic with trace elements is about 2.5 to 4.5 times higher. Animal test results show that when the calcium phosphate ceramic containing trace elements of the present invention is used, a large area of new bone formation and many Haversian canals are observed in the bone defect site. Almost all calcium phosphate ceramic particles are surrounded by the new bone tissue, proving that the calcium phosphate ceramic containing trace elements of the present invention indeed has excellent osteoinductive activity. On the contrary, when commercially available bone grafting materials without trace elements are used, many fat cells appear in the bone defect area and cannot effectively promote the formation of new bone tissue.

The calcium phosphate ceramic of the present invention is prepared from calcium-containing biological wastes, such as oyster shells, egg shells, abalone shells, mussel shells, etc. The calcium phosphate ceramic contains a variety of trace elements, including at least Mg, Sr, Na and Fe. It is not required to incorporate ions artificially. These trace elements are necessary for the growth of human bones. Compared with apatite synthesized by traditional chemicals, they have better biocompatibility and can reduce the rejection with human soft tissues such as skin, muscles and gums. It becomes an ideal component for orthopedic and dental implant materials.

The calcium phosphate ceramic of the present invention has high porosity and porous structure. Its interconnected porous structure is similar to the structure of natural bone, which means that bone cells can grow within the porous structure. Moreover, the calcium phosphate ceramic of the present invention has high biocompatibility and can interact with living cells and tissues, which can ensure cells to attach, proliferate and differentiate on the surface of the calcium phosphate ceramic. Additionally, the porous structure of calcium phosphate ceramic materials can promote cell migration, angiogenesis, and provide excellent structural support at the site of its implantation.

An ideal bone filling material should be easily degraded from the body without hindering the formation of new tissue, and its degradation products must be non-toxic. The calcium phosphate ceramic of the present invention has good biodegradability, and its degradation rate is synchronized with the rate of new bone tissue formation. If it degrades too fast, it will cause holes in the bone defect area. If it degrades too slowly, it will hinder the growth of new tissue.

### BRIEF DESCRIPTION OF THE SEVERAL VIEWS OF THE DRAWINGS

FIG. 1 shows X-ray diffraction (XRD) analysis diagrams of hydroxyapatite (upper) and biphasic calcium phosphate ceramics (lower).

FIG. 2 shows Fourier-transform infrared spectroscopy (FTIR) analysis images of hydroxyapatite (upper) and biphasic calcium phosphate ceramics (lower).

FIGS. 3A and 3B show scanning electron microscopy (SEM) images of biphasic calcium phosphate ceramics at low magnification (FIG. 3A) and high magnification (FIG. 3B).

FIG. 4 shows an EDS analysis spectrum of the biphasic calcium phosphate ceramics.

FIG. 5 shows a pore size distribution of the biphasic calcium phosphate.

FIG. 6 shows the cell morphology of MG-63 cells cultured with the extraction medium of the biphasic calcium phosphate ceramic or commercially available bone graft material (Bicera) for 1, 4 and 7 days, observed through an optical microscope, in which phenol was used as a positive control group and alumina was used as a negative control group.

FIG. 7 shows the cell morphology of MG-63 cells cultured with the extraction medium of the biphasic calcium phosphate ceramic or commercially available bone graft material (Bicera) for 1, 4 and 7 days. Cells were stained using ActinGreen™ 488 Ready Probes® and observed through a fluorescence microscope, in which phenol was used as a positive control group and alumina was used as a negative control group.

FIG. 8 shows the cell viability of MG-63 cells cultured with the extraction medium of the biphasic calcium phosphate ceramic or commercially available bone graft material (Bicera) for 1, 4 and 7 days by WST-1 assay, in which phenol was used as a positive control group and alumina was used as a negative control group. The O.D. value of the positive control group is 0. Statistically significant (*) is set at a p-value of <0.05, and highly significant (**) as p<0.005.

FIG. 9 shows alkaline phosphatase (ALP) activity of MG-63 cells cultured with the extraction medium of the biphasic calcium phosphate ceramic or commercially available bone graft material (Bicera) for 7, 14 and 21 days, in which phenol was used as a positive control group and alumina was used as a negative control group. The O.D. value of the positive control group is 0. Statistically significant (*) is set at a p-value of <0.05, and highly significant (**) as p<0.005.

FIGS. 10A and 10B show the photographs of the bone defect area of the femur of a New Zealand white rabbit at 24 weeks after being implanted with the commercially available bone graft material (Bicera) (FIG. 10A) and the biphasic calcium phosphate ceramic of the present invention (FIG. 10B). The positions marked by the circles are the surgical area, showing the healing of the outer cortical bone.

FIGS. 11A, 11B, 11C and 11D show histological section images of the bone defect area of the femur of New Zealand white rabbits at 24 weeks after being implanted with commercially available bone graft materials (Bicera) (FIGS. 11A and 11B) and biphasic calcium phosphate ceramics of the present invention (FIGS. 11C and 11D). Many Haversian systems can be observed in the group of biphasic calcium phosphate ceramics of the present invention, which are the main morphological functional units of compact bone tissue. The black dotted line represents the junction of the host bone and the defect area. OB indicates the host bone, and NB indicates the new bone.

FIGS. 12A and 12B show Micro-CT images of the bone defect area of the femur of a New Zealand white rabbit at 24 weeks after being implanted with commercially available bone graft material (Bicera) (FIG. 12A) and the biphasic calcium phosphate ceramic of the present invention (FIG. 12B), showing the degree of healing.

FIG. 13 shows quantitative results of micro-CT image analysis of the bone defect area of the femur of a New Zealand white rabbit at 24 weeks after being implanted commercially available bone graft material (Bicera) and the biphasic calcium phosphate ceramic of the present invention. The left axis represents the ratio of bone volume to total volume (BV/TV), and the right axis represents bone thickness (mm).

### DETAILED DESCRIPTION

The following discloses the embodiments of the present invention, which does not limit the present invention to be implemented in the following aspects. It is intended to illustrate the details and implementation effects of the present invention.

### Preparation of Hydroxyapatite

Eggshells are washed and dried, then grinded to obtain eggshell powder. Mix weight percentage 12% of eggshell powder, 16% of acetic acid and the remaining weight percentage of water to dissolve the eggshells, and add 10 vol % of aqueous solution of diammonium hydrogen phosphate to obtain a suspension after mixing; a pH value of the suspension is adjusted to 10 (it can be adjusted to strong alkali or weak alkali solution) by evenly mixing with an ammonia solution to obtain a uniformly-mixed alkaline solution. The uniformly-mixed alkaline solution is subjected to precipitation reaction at room temperature for approximately 6 to 18 hours. After precipitation, the uniformly-mixed alkaline solution is centrifuged to obtain a precipitate. This step can also be done by using suction filtration to obtain the precipitate. Dry and grind the precipitate to obtain hydroxyapatite powder.

### Preparation of Biphasic Calcium Phosphate Ceramic

Mix the above hydroxyapatite powder with 40 vol % stearic acid. Stearic acid is used as a pore-forming agent to create pores in the sample. After mixing evenly, pour it into a cylindrical mold and cold-press it at 200 MPa into a cylindrical green compact (diameter 5 mm, height 7.5 mm). Place the green compact in a high-temperature furnace for sintering, wherein the temperature is raised to 1,000° C. at a heating rate of 5 degrees per minute and this temperature is maintained for 3 hours. Next, the temperature is raised to 1,300° C. at a heating rate of 2 degrees per minute and this temperature is maintained for 25 hours. Finally, it is cooled to room temperature in the furnace to obtain a biphasic calcium phosphate ceramic.

The apparent density of the biphasic calcium phosphate ceramic was calculated using the Archimedes' principle. The porosity was then calculated by as follows:

$$Porosity = ( 1 - Apparent density / ρ ) \times 100 \%$$

where ρ is the theoretical density of the biphasic calcium phosphate ceramic based on a theoretical density for hydroxyapatite of 3.16 g/cm³ and a theoretical density for β-tricalcium phosphate of 3.07 g/cm³.

Using X-ray diffraction (XRD) analyzer to analyze the phase composition of the above hydroxyapatite powder and biphasic calcium phosphate ceramics. The detection conditions are: voltage and current are 40 kV and 40 mA, using Cu Kα radiation (λ=0.15406 nm), over the 2θ range of 20 to 50° at a scanning rate of 2°/min. Calculate the crystallinity (X_{c}) of hydroxyapatite using the following formula:

$${X}_{C} = 1 - ( {V}_{112 / 300} / {I}_{300} )$$

V_{112/300}: The intensity of the hollow between the (112) and (300) diffraction peaks; I₃₀₀: The intensity at the (300) diffraction peak.

Calculate the crystallite size of hydroxyapatite phase from the full width at half maximum (FWHM) of the diffraction peak of the (002) plane along the C-axis direction and using the following Debye-Scherrer formula:

$$D = K λ / ( F W H M \times cos θ )$$

D: Crystallite size (nm); K: dimensionless shape factor (K=0.94); λ: Wavelength of X-ray beam (λ=0.15406 nm), and θ: Bragg's angle) (°).

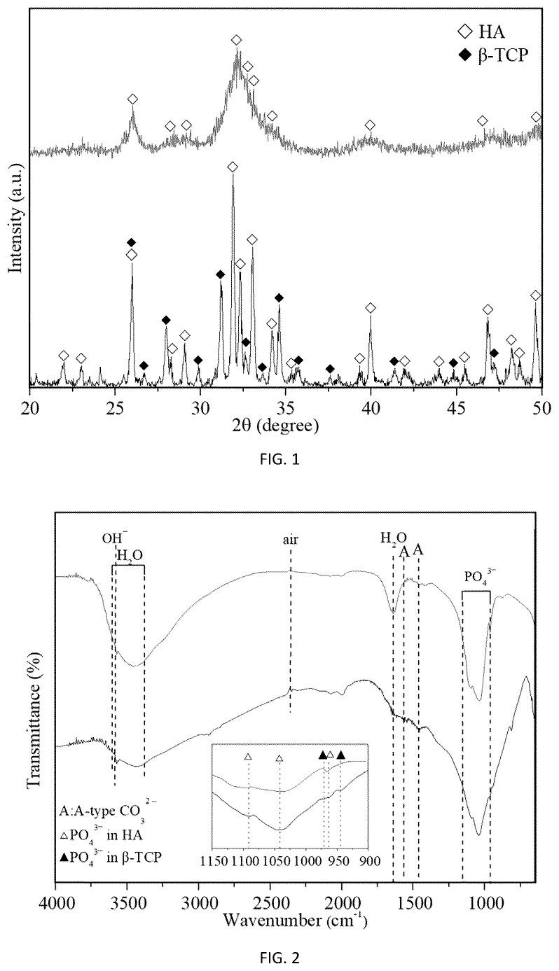

The XRD analysis results are shown in FIG. 1. The XRD spectrum in the upper part of FIG. 1 shows that the prepared hydroxyapatite powder is single-phase hydroxyapatite. There is no raw material residue in the powder, and its crystallinity is 25%. Using the Debye-Scherrer formula to calculate, the crystallite size is approximately 39.02 nm. The XRD spectrum in the lower part of FIG. 1 confirms that the above-mentioned sintered product is a biphasic calcium phosphate material containing hydroxyapatite and β-tricalcium phosphate, wherein the volume ratio of hydroxyapatite to β-tricalcium phosphate is approximately 60:40. Use pure-phase hydroxyapatite and β-tricalcium phosphate powder to prepare different proportions of biphasic HA/β-TCP mixtures. Through the XRD analysis results of these biphasic HA/β-TCP materials, the volume ratio of hydroxyapatite to β-tricalcium phosphate in biphasic calcium phosphate ceramics of the present invention is calibrated.

Use FTIR to analyze the above-mentioned hydroxyapatite powder and biphasic calcium phosphate ceramics, and observe the functional groups in the wave number range of 4,000 cm⁻¹ to 450 cm⁻¹. The analysis results are shown in FIG. 2. The FTIR spectrum in the upper part of FIG. 2 further confirms that the prepared hydroxyapatite powder is indeed single-phase hydroxyapatite. The lower part of FIG. 2 and Table 1 show the FTIR spectrum of the above-mentioned biphasic calcium phosphate ceramics. The peaks at 961 cm⁻¹, 1044 cm⁻¹ and 1089 cm⁻¹ correspond to the PO₄³⁻ functional group of HA, while the peaks at 945 cm⁻¹ and 969 cm⁻¹ correspond to the PO₄³⁻ functional group of β-TCP. The peak at 3574 cm⁻¹ is the characteristic peak of the OH⁻ group of HA, and the peaks at 1460 cm⁻¹ and 1551 cm⁻¹ represent A-type CO₃²⁻ group. Carbonated hydroxyapatite is chemically similar to human bones and was found to be more effective in a shorter postoperative rehabilitation program. Table 1 summarizes the characteristic peaks of functional groups from the HA and β-TCP in the biphasic calcium phosphate ceramics of the present invention and references.

TABLE 1
The characteristic peaks of functional groups from
HA and β-TCP in the biphasic calcium phosphate
ceramics of the present invention and references.

| | Observed vibrational frequencies (cm⁻¹) | | |
|---|---|---|---|
| | Biphasic calcium | | |
| | phosphate ceramic of the | | |
| Functional groups | present invention | Reference 1 | Reference 2 |
| PO₄³⁻ in HA | 961 | 963 | 965 |
| | 1044 | 1045 | 1041 |
| | 1089 | 1090 | 1089 |
| PO₄³⁻ in β-TCP | 945 | — | 950 |
| | 969 | — | 965 |
| CO₃²⁻ | 1460 | 1462 | — |
| | 1551 | — | — |
| OH⁻ | 3574 | 3572 | 3572 |

Reference 1: Cox, S. C. et al. (2013) ‘Low temperature aqueous precipitation of needle-like nanophase hydroxyapatite’, Journal of Materials Science: Materials in Medicine, 25(1), pp. 37-46.
Reference 2: Lee, S. et al. (2022) ‘Microhardness and microstructural properties of a mixture of hydroxyapatite and β-tricalcium phosphate’, Journal of Asian Ceramic Societies, 11(1), pp. 11-17.

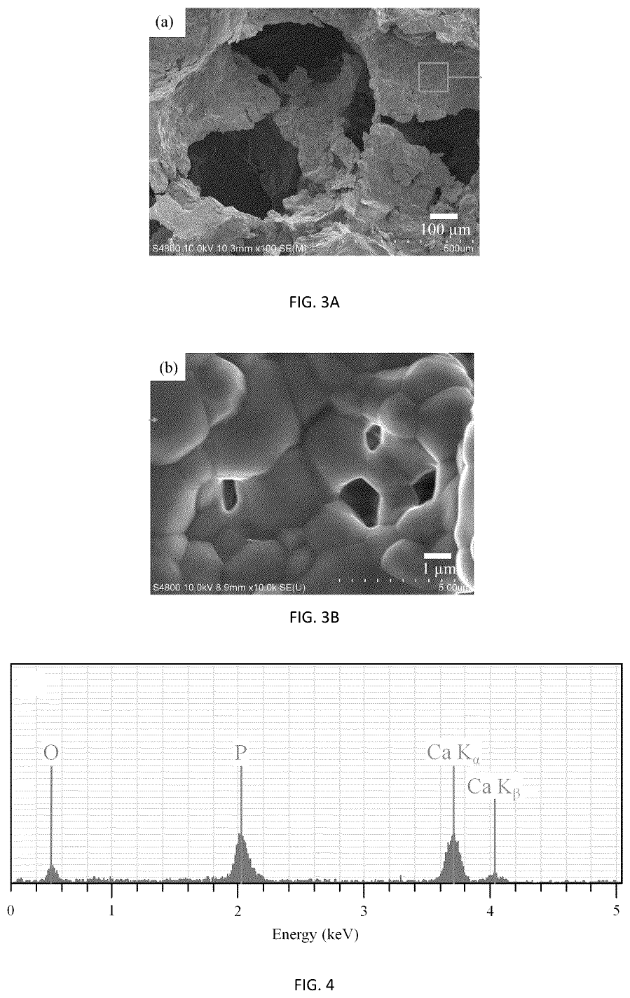

Next, a scanning electron microscope was used to observe the porous structure of the above-mentioned biphasic calcium phosphate ceramic, as shown in FIGS. 3A and 3B, which show that the above-mentioned biphasic calcium phosphate ceramic has uniformly dispersed pores, including open pores and closed pores. FIG. 3A low magnification observation results show that the above-mentioned biphasic calcium phosphate ceramic has many pores larger than 100 μm, and the pores are interconnected. The pores on one side can be observed from the pores on the other side. The partially enlarged observation result of FIG. 3A is shown in FIG. 3B, which shows that the above-mentioned biphasic calcium phosphate ceramic also has many pores smaller than 10 μm. The pore size of the above-mentioned biphasic calcium phosphate ceramic ranges from 0 to 850 μm, the porosity is 53%, and the porosity of the open pores is approximately 38%. The open pores help the migration and proliferation of new bone tissue and angiogenesis.

The inorganic components of the above-mentioned biphasic calcium phosphate ceramics were analyzed using SEM-EDS and ICP-MS methods. As shown in FIG. 4, the calcium-phosphorus ratio (Ca/P) of the above-mentioned biphasic calcium phosphate ceramics is approximately 1.70. Table 2 shows that the above-mentioned biphasic calcium phosphate ceramic contains a variety of trace elements, including Mg ion 3951 ppm, Sr ion 399 ppm, Na ion 1022 ppm, Fe ion 37 ppm, and does not contain heavy metal elements.

TABLE 2
The concentrations of trace elements of the biphasic
calcium phosphate ceramics of the present invention.

| Element | Mg | Sr | Na | Fe | Pb | Hg | As | Cd |
|---|---|---|---|---|---|---|---|---|
| Concentration | 3951 | 399 | 1022 | 37 | Not | Not | Not | Not |
| (ppm) | | | | | de- | de- | de- | de- |
| | | | | | tected | tected | tected | tected |

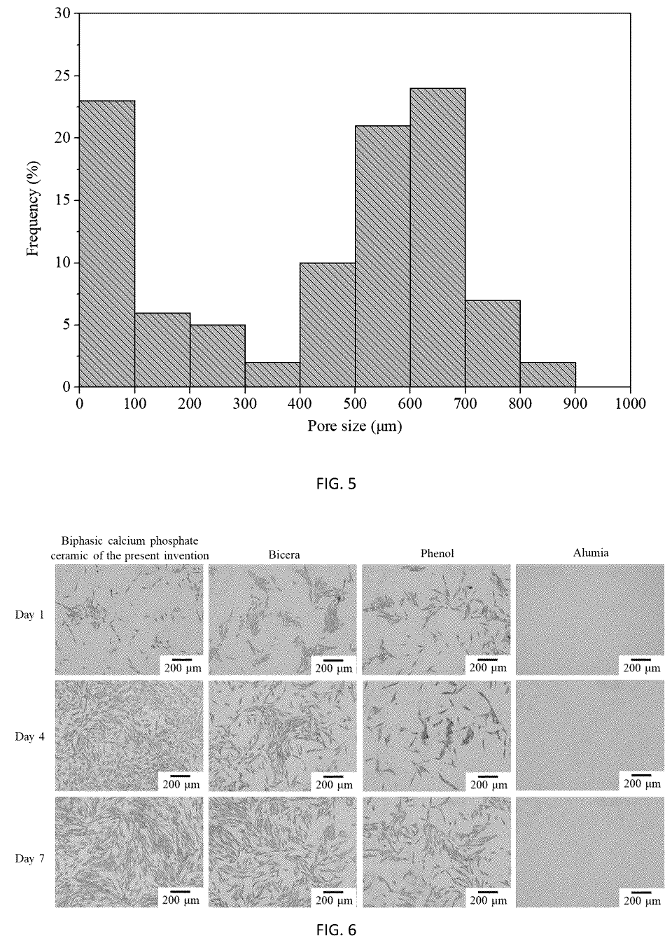

Take multiple SEM photos at a magnification of 30× to characterize the pore size of the biphasic calcium phosphate ceramics, and randomly select 100 pores from multiple photos to calculate the pore size distribution of the biphasic calcium phosphate ceramics. The pore size distribution is shown in FIG. 5 and Table 3, the pore size of the above-mentioned biphasic calcium phosphate ceramic ranges from 0 to 850 μm, and approximately 80% of the pores have a pore size greater than 100 μm. The pore size must be at least larger than 100 μm to allow cell migration, and pores larger than 300 μm can promote angiogenesis and the formation of new bone tissue. The above-mentioned biphasic calcium phosphate ceramics also contain many pores smaller than 10 μm, providing a larger surface area, increase protein adsorption, ion exchange and formation of mineralized tissue. It can also provide nutrition and osteogenesis related signaling molecules to facilitate interaction between cells and the implanted graft, promote cell proliferation and accelerate bone growth.

TABLE 3
The pore size distribution of the biphasic calcium
phosphate ceramic of the present invention.

| Pore size (μm) | Percentage of pores (%) |
|---|---|
| 0-100 | 23% |
| 100-200 | 6% |
| 200-300 | 5% |
| 300-400 | 2% |
| 400-500 | 10% |
| 500-600 | 21% |
| 600-700 | 24% |
| 700-800 | 7% |
| 800-900 | 2% |

### EXAMPLES

### Example 1: Effects of Calcium Phosphate Ceramics of the Present Invention on Cell Proliferation and Cell Morphology

The biphasic calcium phosphate material of the present invention is pulverized into granules with a particle size of about 0.5-1.0 mm, and the biphasic calcium phosphate ceramic granules are soaked into the cell culture medium at a ratio of 0.2 g/ml to obtain the extract of biphasic calcium phosphate ceramics. Next, the commercially available artificial bone graft material Bicera (purchased from Wiltrom Co., Ltd.) was used as a control group without trace elements. Bicera is a biphasic calcium phosphate material composed of 60% HA and 40% β-TCP.

The MG-63 cell line was added to a 24-well plate at a concentration of 1×10⁴ cells/well, and cell culture medium containing the extract of the biphasic calcium phosphate ceramic of the present invention or the Bicera extract (100 μl/well) was added for cell culture. Cell morphology was observed after culturing for 1, 4, and 7 days, with phenol as the positive control group and alumina as the negative control group. Test results are shown in FIG. 6, the number of cells increased with an increase in the culture time for both Bicera and the biphasic calcium phosphate ceramic of the present invention. After culturing for 4 days, the cell number in the biphasic calcium phosphate ceramic group of the present invention increased significantly, implicating that the biphasic calcium phosphate ceramic of the present invention has the potential to promote cell proliferation. After culturing for 7 days, the number of cells further increased, and the biphasic calcium phosphate ceramic group of the present invention has a more elongated cell morphology, which can promote cell differentiation.

Experimental results show that the biphasic calcium phosphate ceramics of the present invention have ideal cell viability and will not cause cytotoxic reactions. Compared with commercially available bone graft materials that do not contain trace elements, the biphasic calcium phosphate ceramics of the present invention have better cell proliferation ability.

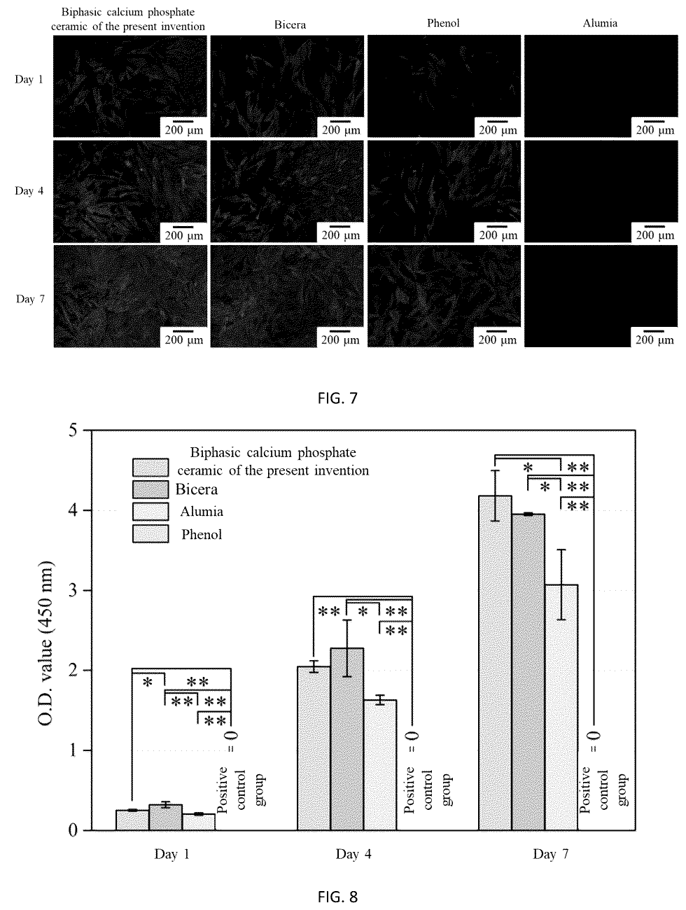

ActinGreen™ 488 Ready Probes® was used to stain the cells, and the attachment and extension of the cell filopodia were observed through a fluorescence microscope. The results are shown in FIG. 7. Compared with Bicera, the biphasic calcium phosphate ceramics of the present invention exhibited strong cortical actin staining after culturing for 4 days. In addition, the cells had clear cytoplasmic actin skeletons. After cell culture for 7 days, the number of cells in the biphasic calcium phosphate ceramic group of the present invention significantly increased with dense and continuous cell sheets. Remarkable flattened and extended cell morphology was observed, indicating that the cells were well attached and had proliferated rapidly.

### Example 2: Cell Viability Test

MG-63 cells were cultured with the extraction medium of the biphasic calcium phosphate ceramic of the present invention or Bicera. After culturing for 1, 4 and 7 days, the cell viability was analyzed through the WST-1 assay, in which phenol was used as a positive control group and alumina served as the negative control group. Results are shown in FIG. 8. The biphasic calcium phosphate ceramic of the present invention and the Bicera group both showed a steady increase over culture time. However, the cell viability of the biphasic calcium phosphate ceramic of the present invention group is significantly higher than those of the other groups after culturing for 7 days. Compared with the phenol in the positive control group and the alumina in the negative control group, the biphasic calcium phosphate ceramic of the present invention has good biocompatibility and is no cytotoxicity.

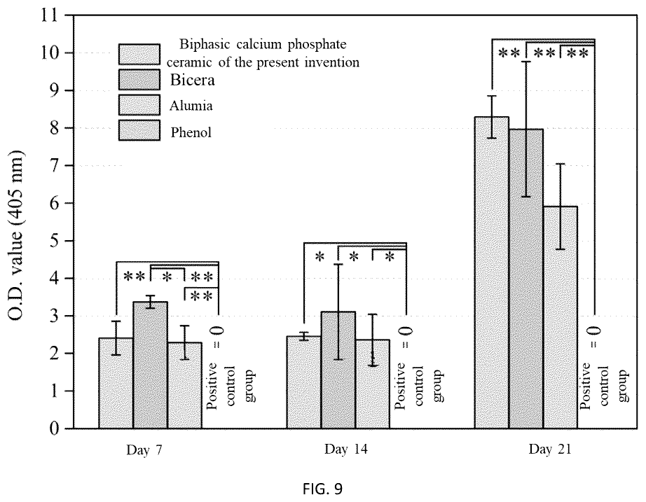

Alkaline Phosphatase (ALP) activity is regarded as a biochemical marker for relatively early osteoblast differentiation. MG-63 cells were cultured with the extraction medium of Bicera or the biphasic calcium phosphate ceramic of the present invention. After culturing for 7, 14 and 21 days, the ALP activity of the MG-63 cells was detected, in which phenol was used as the positive control group and alumina was used as the negative control group. The experimental results are shown in FIG. 9. On the 7th day of cell culturing observation, the ALP activity of MG-63 cells on the Bicera was significantly higher than those of the other groups. On the day 21, the ALP activities of the biphasic calcium phosphate ceramic of the present invention and Bicera were higher than those of the positive groups. However, the ALP activities of the biphasic calcium phosphate ceramic of the present invention and Bicera were not significantly different at day 7, 14, and 21.

### Example 3: Animal Experiments

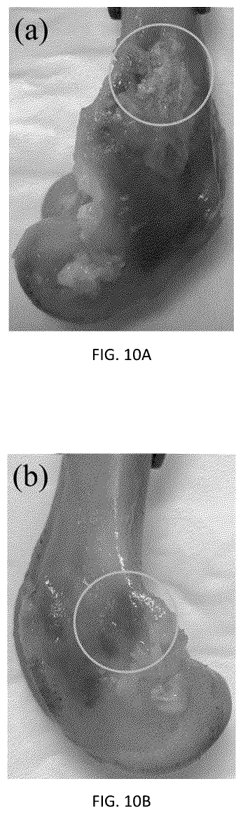

The biphasic calcium phosphate ceramic of the present invention or Bicera was implanted into the femur of a New Zealand white rabbit. After 24 weeks of implantation, the photos of femurs containing the implantation area are shown in FIGS. 10A and 10B. FIG. 10B shows the bone defect area where the biphasic calcium phosphate ceramic was implanted has been completely repaired by new bone, showing that the biphasic calcium phosphate ceramic of the present invention has good healing effect. Moreover, the implanted white calcium phosphate ceramic is no longer visible in the defect area. In contrast, the bone defect area where Bicera was implanted subsided, as shown in FIG. 10A, indicating that the amount of new bone was insufficient and the wound was not fully healed.

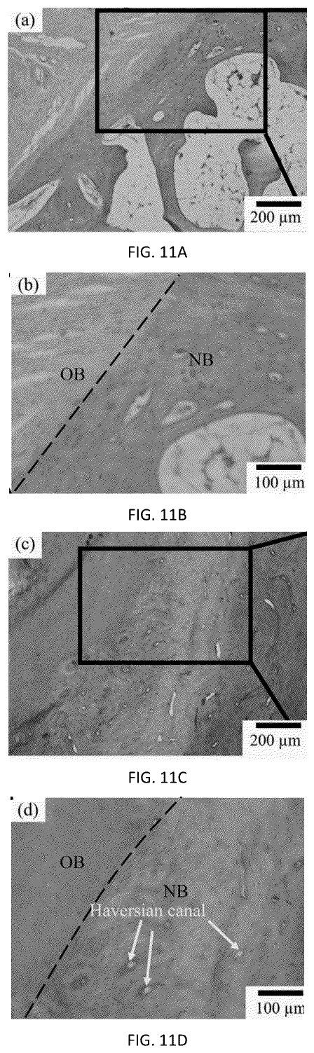

The biphasic calcium phosphate ceramic of the present invention or Bicera was implanted into the bone defect area of New Zealand white rabbit. After 24 weeks, sectioning and H & E staining were performed. The experimental results are shown in FIGS. 11A, 11B, 11C and 11D. In the group implanted with Bicera, as shown in FIGS. 11A and 11B, new bone has grown into the defect area, but in the group implanted with Bicera, many fat cells were observed in the defect area, and fat cells hinder the growth of new bone tissue. On the contrary, in the group implanted with the biphasic calcium phosphate ceramics of the present invention, as shown in FIGS. 11C and 11D, the density of new bone is higher, and many Haversian canals can be observed.

Experimental results show that the biphasic calcium phosphate ceramic of the present invention has a larger new bone formation area in the bone defect area than commercially available bone graft materials.

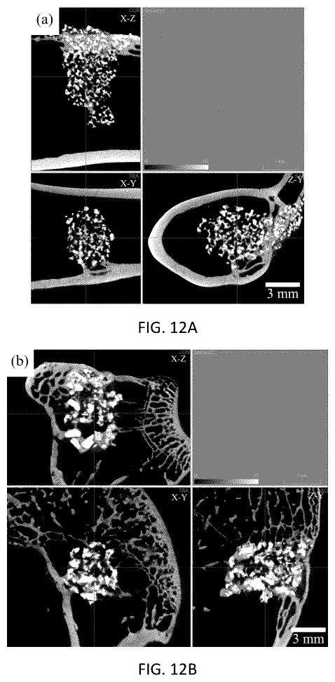

FIGS. 12A and 12B show the micro-CT images at 24 weeks after implantation with Bicera or the biphasic calcium phosphate ceramic of the present invention. The white part in the image represents the implanted bone graft material, and the gray part represents the bone tissue. The experimental results show that both the biphasic calcium phosphate ceramic of the present invention and Bicera groups had residual bone graft material, but area covered by the residual bone graft material in the Bicera group is larger. Moreover, in the biphasic calcium phosphate ceramic of the present invention group, almost all implanted calcium phosphate ceramic granules are surrounded by new bone tissue, proving that the calcium phosphate ceramic of the present invention can promote osteogenesis faster and more effectively.

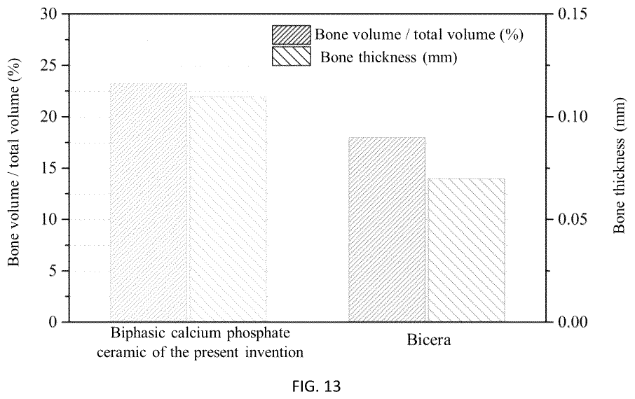

The quantitative results of micro-CT images are shown in FIG. 13, showing that the biphasic calcium phosphate ceramic of the present invention has a higher BV/TV ratio and greater bone thickness than Bicera.

What is claimed is:

1. A calcium phosphate ceramic with trace elements, wherein a main component of the calcium phosphate ceramic is selected from a group consisting of hydroxyapatite, α-tricalcium phosphate, β-tricalcium phosphate, tetracalcium phosphate and any combination thereof;
the calcium phosphate ceramic comprises various trace elements, at least including Mg and Sr.

2. The calcium phosphate ceramic of claim 1, wherein the main component of the calcium phosphate ceramic is a biphasic calcium phosphate composed of hydroxyapatite and β-tricalcium phosphate.

3. The calcium phosphate ceramic of claim 1, wherein an amount of the Mg is 1,000˜10,000 ppm.

4. The calcium phosphate ceramic of claim 1, wherein an amount of the Sr is 100˜1,000 ppm.

5. The calcium phosphate ceramic of claim 1, wherein the calcium phosphate ceramic has a porosity ranging from 50% to 90%.

6. The calcium phosphate ceramic of claim 1, wherein each pore of the calcium phosphate ceramic ranging has a pore size ranging from 0 to 1,000 μm, and at least 70% of the pores have a pore size greater than 100 μm.

7. A bone repair material, comprising the calcium phosphate ceramic of claim 1.

8. Use of the calcium phosphate ceramic of claim 1, wherein the calcium phosphate ceramic is used to prepare dental repair materials, orthopedic repair materials, drug carrier materials or tissue engineering scaffolds.
